(19) United States
(12) Patent Application Publication
(10) Pub. No.: US 2026/0076807 A1
(43) Pub. Date: Mar. 19, 2026

(54) Plate for Surgical Fusion Design for Transnasal Placement

(71) Applicant: University of Cincinnati, Cincinnati, OH (US)

(72) Inventors: Joel Kaye, Cincinnati, OH (US); Jonathan Forbes, Cincinnati, OH (US)

(73) Assignee: University of Cincinnati, Cincinnati, OH (US)

(21) Appl. No.: 19/330,156

(22) Filed: Sep. 16, 2025

Related U.S. Application Data
(60) Provisional application No. 63/695,345, filed on Sep. 16, 2024.

(51) Int. Cl. A61F 2/44; A61B 17/70; A61F 2/30; A61L 27/06

(52) U.S. Cl. CPC A61F 2/447; A61B 17/7059; A61F 2/30749; A61L 27/06; A61F 2310/00023; A61L 2430/02

(57) ABSTRACT

An implant system for the fusion of the occipitocervical joint is provided. The system involves a pair of plates including a right plate and a left plate. The right plate has a first right plate opening and a second right plate opening. The left plate has a first left plate opening and a second left plate opening. The system also includes at least four bone fixation fasteners. Two fasteners are engageable with the plates and capable of fixing with the occipital condyle. The other two fasteners are engageable with the plates and capable of fixing with the C1 lateral mass.

### CROSS-REFERENCE TO RELATED APPLICATIONS

This application claims priority to U.S. Provisional Application Ser. No. 63/695,345, filed Sep. 16, 2024, the contents of which is hereby incorporated by reference in its entirety.

### TECHNICAL FIELD

The present invention relates to methods for surgical fusion.

### BACKGROUND OF THE INVENTION

Irreducible ventral compression of the brainstem or upper cervical spinal cord can result from several pathologic processes unique to the craniovertebral junction (CVJ), affecting both adult and pediatric patients. These include, but are not limited to, tumors, trauma, rheumatoid arthritis, infection, and congenital anomalies (Chiari 1 malformation, basilar invagination). Patients may present with lower cranial neuropathies, sensory deficits, and myelopathy—and the development of symptoms represents a strong indication for decompressive surgery. Irrespective of the pathology being treated, ventral decompressive surgery of the CVJ will almost always result in some degree of craniocervical instability.

Over the past 20 years, considerable progress has been made in expanding the utility of the endoscopic endonasal approach (EEA) to the CVJ, which is now routinely employed to address a variety of pathologies. Commonly accepted indications for this approach include odontoidectomy for basilar invagination and resection of tumors that occupy the CVJ and/or basiocciput. However, the vast majority of patients who undergo ventral odontoidectomy, and a smaller yet considerable subset of patients who undergo endonasal tumor resection, will require subsequent occipitocervical fixation (OCF) and fusion. Currently, this is performed through a separate-stage posterior approach which, despite high fusion rates, is associated with extensive muscle dissection, postoperative pain, blood loss, and risk of injury to the vertebral arteries.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

In a first embodiment of the invention, an implant system for the fusion of the occipitocervical (O-C1) joint is provided. The system involves a pair of plates including a right plate and a left plate. The right plate has at least a first right plate opening and a second right plate opening. The left plate has at least a first left plate opening and a second left plate opening. The system also includes at least four bone fixation fasteners. A first fastener is disposed with the first right plate opening. A second fastener is disposed with the first left plate opening. A third fastener is disposed with the second right plate opening. A fourth fastener is disposed with the second left plate opening. Also, the first fastener is engageable with the right plate and capable of fixing with the occipital condyle. The second fastener is engageable with the left plate and capable of fixing with the occipital condyle. The third fastener is engageable with the right plate and capable of fixing with the C1 lateral mass. The fourth fastener is engageable with the left plate and capable of fixing with the C1 lateral mass.

In one embodiment, the pair of plates can be a material such as carbon fiber, polylactic acid, stainless steel alloys, commercially pure titanium, titanium alloys, ceramics, thermoplastics, polyethylene terephthalate (PET), fabric, silicone, polyurethane, silicone-polyurethane copolymers, polymeric rubbers, polyolefin rubbers, hydrogels, semi-rigid and rigid materials. In another embodiment, the pair of plates are titanium alloy.

In one embodiment, the bone fixation fasteners are selected from the group consisting of bone screws, helical nails, distally expanding nails, and distally expanding screws. In another embodiment, the bone fixation fasteners are bone screws. In one embodiment, the right plate and left plate each have an average thickness of 2.5 mm.

In another embodiment, an atlanto-occipital fusion method for fixating a 0-C1 joint is provided. The method involves preparing an endonasal surgical path using a binostril approach. This involves removing the inferior portion of the posterior septum, performing a myomucosal flap incision to create a flap, retracting the flap to expose the O-C1 joint, bilaterally decorticating the O-C1 joint, and inserting a plate into each side of the decorticated 0-C1 joint; wherein the plate is part of the implant system described above.

In one embodiment, the pair of plates can be a material such as carbon fiber, polylactic acid, stainless steel alloys, commercially pure titanium, titanium alloys, ceramics, thermoplastics, polyethylene terephthalate (PET), fabric, silicone, polyurethane, silicone-polyurethane copolymers, polymeric rubbers, polyolefin rubbers, hydrogels, semi-rigid and rigid materials. In another embodiment, the pair of plates are titanium alloy.

In one embodiment, the bone fixation fasteners are selected from the group consisting of bone screws, helical nails, distally expanding nails, and distally expanding screws. In another embodiment, the bone fixation fasteners are bone screws. In one embodiment, the right plate and left plate each have an average thickness of 2.5 mm.

### BRIEF DESCRIPTION OF THE DRAWINGS

The objects and advantages of the disclosed invention will be further appreciated in light of the following detailed descriptions and drawings in which:

FIG. 1 is a series of images showing 3D segmentation of an O-C1 joint. A thin-cut computed tomography scan of a cadaveric head is uploaded to 3D Slicer and the region of interest is selected slice-by-slice in the axial, sagittal, and coronal planes. The resultant 3D construction can be seen in the top right quadrant.

FIGS. 2A-2E are a series of images showing a plate according to the present invention being designed in sequential steps using computer-aided design software. The 3D-segmented O-C1 joint is imported into Blender (FIG. 2A). Plates are designed to span each O-C1 joint and contoured to sit flush onto the bone surface (FIG. 2B). Countersunk screw holes are created in the plates at each starting point (FIG. 2C) and designed to accommodate appropriate screw trajectories (FIG. 2D). The plates and O-C1 joint are 3D-printed in polylactic acid for prototyping and finally, 3D printed in the titanium alloy Ti-6A1-4V (FIG. 2E).

FIGS. 3A-3D are a series of intraoperative endoscopic images demonstrating relevant surgical anatomy and procedural steps. Within the posterior nasopharynx (NP), several key anatomic landmarks can be easily visualized: pharyngeal tubercle (PT), Rosenmuller fossa (RF), torus tubarius (TT), and the eustachian tube (ET) entrance (FIG. 3A). An inverted U-shaped nasopharyngeal flap (outlined in white) is raised sharply (FIG. 3B) to expose the occipital condyles (red), C1 anterior arch (black), and odontoid process (yellow) (FIG. 3C). The plate is then introduced, positioned onto the bony surface, and fixated to bone with screws (FIG. 3D).

FIGS. 4A-4D are a series of images. A postfixation endoscopic image demonstrating the final positions of the plates with screws in place is shown in (FIG. 4A). Computed tomography imaging confirms optimal screw placement seen in both the sagittal (FIG. 4B) and axial (FIGS. 4C and 4D) planes.

FIGS. 5A-5E is a series of images showing screw entry sites and trajectories that were measured and recorded. The C1 lateral site-of-entry (FIG. 5A) is measured from the C1 anterior tubercle to the screw entry point (red and green lines represent a sample distance for the right and left screws, respectively)). Screw angulation in the axial plane (FIG. 5B) is measured with respect to the midline (long red line denotes midline; short red line and green line represent screw trajectories on the right and left, respectively). Screw angulation in the sagittal plane is measured with respect to the palatal plane (PP) (FIG. 5C and FIG. 5D). Note: the trajectory of the occipital condyle (OC) screw in Panel C is superior to the PP, thus its angle is denoted with a “,” whereas the trajectory of the C1 screw in Panel D is inferior to the PP, thus its angle is denoted with a “-.” The OC lateral site-of-entry is measured from the medial O-C1 joint (FIG. 5E, horizontal light double arrow). The rostral/caudal site-of-entry is measured from the medial O-C1 joint (FIG. 5E, vertical light double arrow for OC, vertical dark double arrow for C1).

FIGS. 6A-6C are a series of images showing that endonasal O-C2 plate-screw fixation may be achieved with an occipital condyle screw and an anterior C1-2 transarticular screw. Postoperative sagittal computed tomography slices from medial (FIG. 6A) to lateral (FIG. 6C) demonstrating endonasal O-C2 fixation on the right side of a single cadaveric specimen.

FIGS. 7A and 7B are a pair of images showing that to avoid injury, accurate screw placement is crucial. A schematic illustration of the operator's endoscopic endonasal view of the craniovertebral junction with key anatomic structures superimposed (FIG. 7A). The internal carotid artery and vertebral artery both run posterolateral to the eustachian tube. The hypoglossal nerve exits the brainstem and courses laterally within the hypoglossal canal on the upper surface of the occipital condyle. A coronal computed tomography image demonstrates screws within their course and highlights their proximity to critical neurovascular anatomy (FIG. 7B). CN XII, hypoglossal nerve; FT, foramen transversarium; HC, hypoglossal canal; ICA, internal carotid artery; JF, jugular foramen; OC, occipital condyle; VA, vertebral artery.

FIGS. 8A-8E are a series of endoscopic and radiographic images (computed tomography and X-ray) demonstrating the final positions of the plate-screw construct in 5 cadaveric specimens (AeE).

FIGS. 9A-9D are schematics showing alternate views of a right plate according to the present invention.

FIGS. 10A-10D are schematics showing alternate views of a left plate according to the present invention.

### DETAILED DESCRIPTION OF THE INVENTION

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

Certain terminology is used in the following description for convenience only and is not limiting. The words “right”, “left”, “lower” and “upper” designate directions in the drawings to which reference is made. The words “inwardly” or “proximally” and “outwardly” or “distally” refer to directions toward and away from, respectively, the geometric center of the implant and related parts thereof. The words, “anterior”, “posterior”, “superior,” “inferior” and related words and/or phrases designate preferred positions and orientations in the human body to which reference is made and are not meant to be limiting. The terminology includes the above-listed words, derivatives thereof and words of similar import.

Similar reference numerals will be utilized throughout the application to describe similar or the same components of each of the preferred embodiments of the implant described herein and the descriptions will focus on the specific features of the individual embodiments that distinguish the particular embodiment from the others

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, micro discectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, muscle, ligaments, tendons, cartilage and/or bone unless specifically referred to otherwise.

The present invention involves a novel method for occipitocervical fixation (OCF) through the endonasal corridor. The endoscopic endonasal approach (EEA) to the craniovertebral junction (CVJ) was first described in a 2002 cadaveric study and first performed on a patient for odontoidectomy three years later, challenging the historically accepted “gold standard” transoral approach. Since then, the EEA to the CVJ has been further refined and is now used to treat a wide range of pathology. While odontoidectomy remains the surgical gold standard for irreducible ventral compression, many authors now consider the endonasal corridor to be the preferred surgical approach. Likewise, the EEA can be utilized to address notoriously difficult to access tumors of the lower clivus and anterior CVJ (e.g., chordomas, meningiomas, and chondrosarcomas) due to its favorable surgical angle of attack, providing several advantages over the transcranial, transcervical, and transoral approaches for ventral midline lesions above the nasopalatine line with limited lateral extension.

### Biomechanical Considerations and CVJ Instability

As the field's collective experience with the EEA to the CVJ has advanced so too has our understanding of its biomechanical implications. It is well-established that odontoidectomy with C1 arch resection results in clinically significant CVJ instability both at O-C1 and C1-2, thus it has become common practice to perform empiric OCF in conjunction with odontoidectomy. A recent meta-analysis reported that despite emerging techniques of C1 arch and transverse ligament preservation to reduce the degree of instability, 83% of patients who underwent odontoidectomy subsequently underwent OCF. Less is known about oncologic resections, and studies on postoperative CVJ instability after endonasal resection has predominantly focused on the degree of OC resection, with no clear consensus. Results from various cadaveric studies have led to a range of recommendations. Vishteh et al recommend consideration of OCF if more than 50% of 1 condyle is resected, while Perez-Orribo et al recommend OCF for >75% condylar resection. Little et al found statistically significantly increased hypermobility at O-C1 after inferior-third clivectomy. However, they concluded that the modest degree of hypermobility may justify observation. In practice, Kooshkabadi et al published a case series of 212 patients who underwent EEA for lower clival lesions with only 3.3% requiring arthrodesis, citing >75% condyle resection or combination with an open approach as independent risk factors associated with craniocervical instability.

### Current and Investigational Methods of OC Fixation

Stabilization of the CVJ is currently performed almost exclusively from a posterior approach with several available techniques, including plate-screw constructs, sublaminar wiring, trans-articular fixation, and others. Although well-studied and generally well-tolerated, the posterior approach has drawbacks, including an association with increased postoperative pain, additional blood loss, risk of nonunion, injury to the vertebral artery, CSF leak, and specific anatomic limitations (obesity, cervical hyperlordosis). Furthermore, the posterior approach necessitates a second-stage operation if performed with odontoidectomy. Therefore, there have been numerous attempts at better characterizing the anatomic and biomechanical feasibility of various ventral approaches.

Recent cadaveric studies describing anterior transcervical approaches for O-C1 and C1-2 trans-articular screw placement suggest that anterior fixation may be biomechanically equivalent to traditional posterior approaches. Interestingly, results from finite element analyses found that incorporating the OCs (as opposed to C1-2 alone) may be necessary for maintaining CVJ stability after odontoidectomy. Still, the implications of bilateral transcervical incisions, with attendant risks to laryngeal structures, are uncertain. In addition, the surgical corridor used in this method of fixation does not lend itself easily to same-stage ventral decompression with odontoidectomy.

Transoral approaches for fixation of the CVJ have also been investigated. Goel et al reported successful transoral plate-screw fixation from the clivus to the cervical vertebrae after odontoidectomy. Subsequent cadaveric and radiographic studies have sought to better characterize the anatomic feasibility of this and similar techniques. The transoral approach has also been used for plate-screw osteosynthesis and reduction of unstable fractures of the C1 anterior arch with satisfactory rates of bony fusion. While transoral OCF has been successfully performed in practice, the morbidity associated with the transoral approach in general limits its appeal. Advocates for the EEA to the CVJ cite several advantages over the transoral approach, including earlier extubation, decreased rate of dysphagia, earlier oral intake, and shorter hospital stay. In light of this work, the present inventors decided to explore the EEA for CVJ fixation.

### Endoscopic Endonasal OC Fixation: Feasibility and Anatomic Considerations

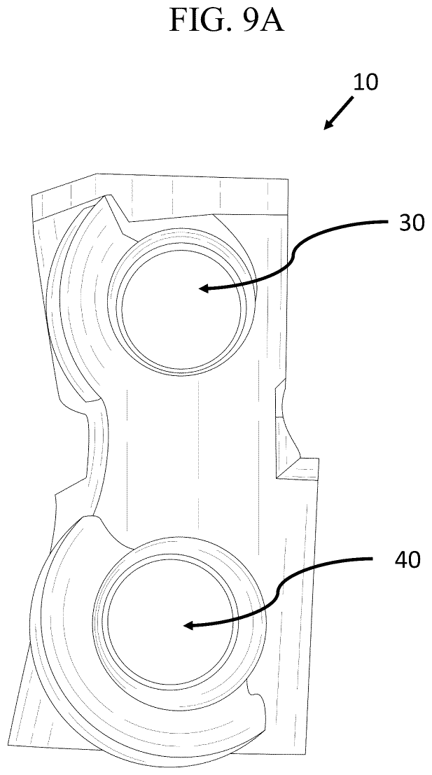

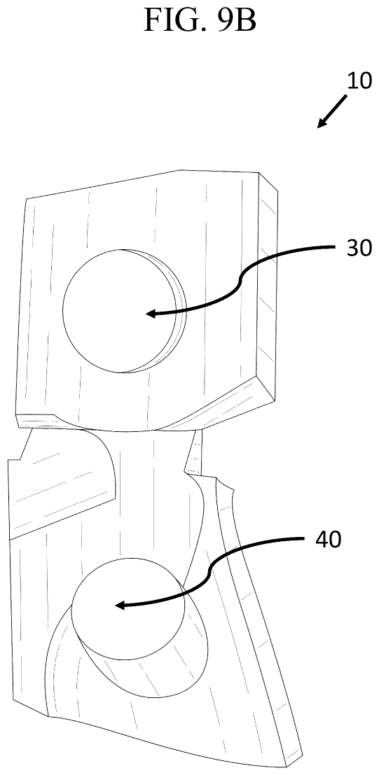

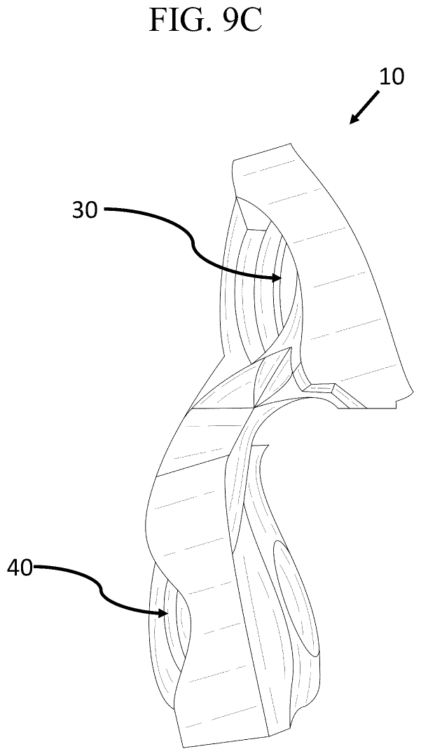

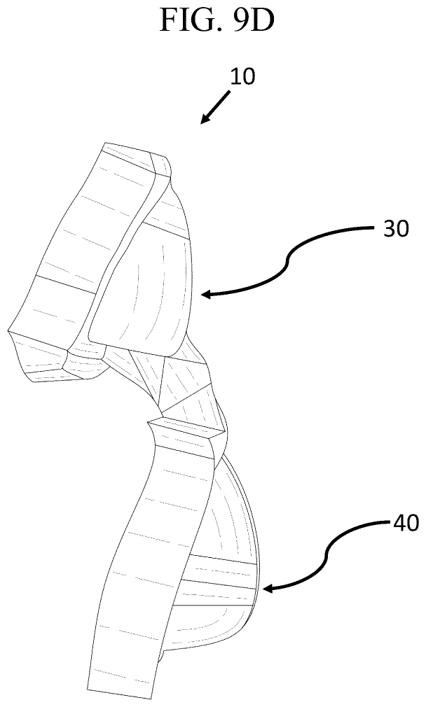

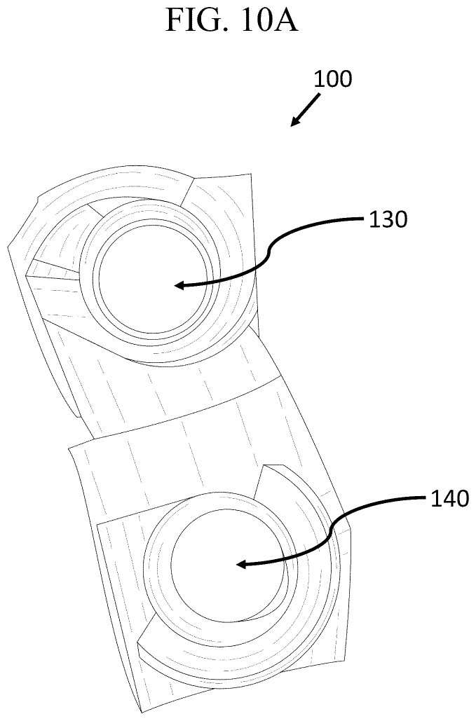

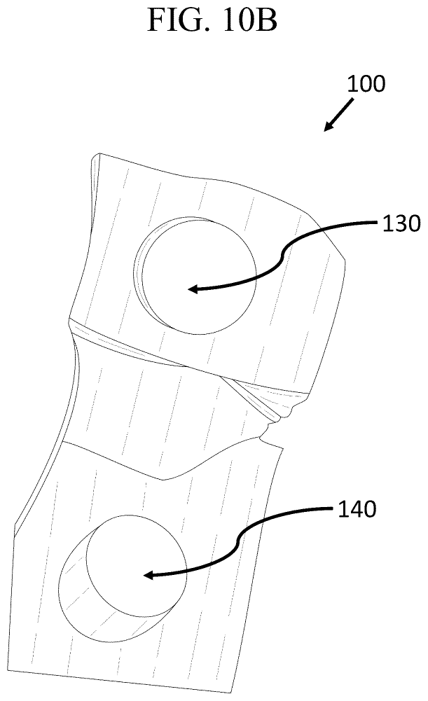

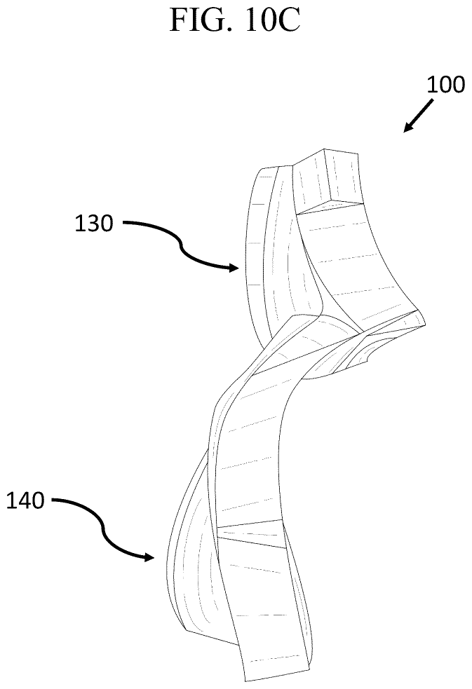

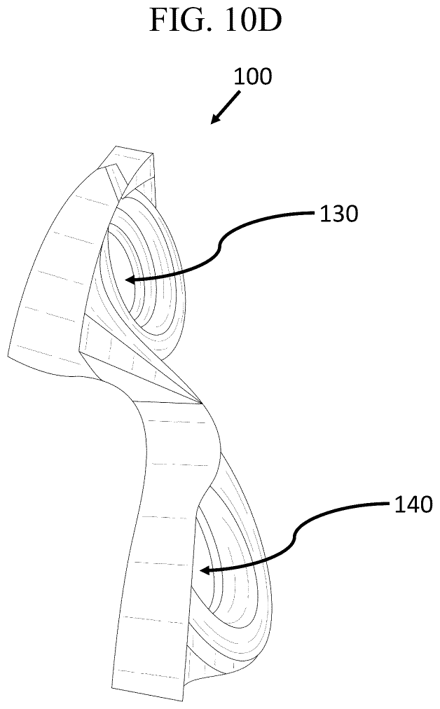

The present invention involves endonasal plate-screw fixation across the craniovertebral junction. A novel construct was successfully designed, 3D printed, and implanted into 5 cadaveric specimens. Referring to FIGS. 9A-9D and 10A-10D, the implant system involves two plates, a right plate (FIGS. 9A-9D) and a left plate (FIGS. 10A-10D). Each plate has two holes and a bone fixation fastener, such as a bone screw, is inserted through each hole. The system uses a total of four screws.

Referring to FIGS. 9A-9D, a right plate 10 includes a first right plate opening 30 and a second right plate opening 40. FIG. 9A shows a front view of the right plate 10. FIG. 9B shows a back view of the right plate 10. FIG. 9C shows a left side view of the right plate 10. FIG. 9D shows a right side view of the right plate 10. In use, a bone fixation fastener is inserted through the first right plate opening 30 and fixes with the C1 lateral mass of a subject. Also, a bone fixation fastener is inserted through the second right plate opening 40 and fixes with the occipital condyle of a subject.

Referring to FIGS. 10A-10D, a left plate 100 includes a first left plate opening 130 and a second left plate opening 140. FIG. 10A shows a front view of the left plate 100. FIG. 10B shows a back view of the left plate 100. FIG. 10C shows a left side view of the left plate 100. FIG. 10D shows a right side view of the left plate 100. In use, a bone fixation fastener is inserted through the first left plate opening 130 and fixes with the C1 lateral mass of a subject. Also, a bone fixation fastener is inserted through the second left plate opening 140 and fixes with the occipital condyle of a subject.

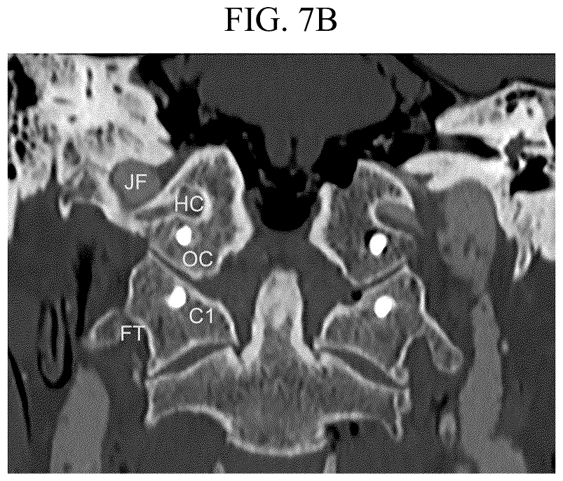

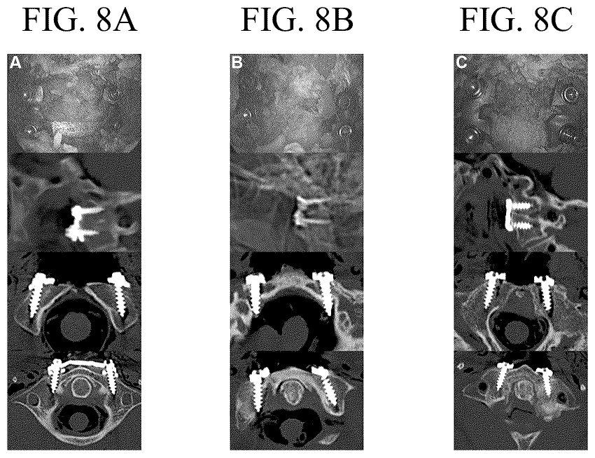

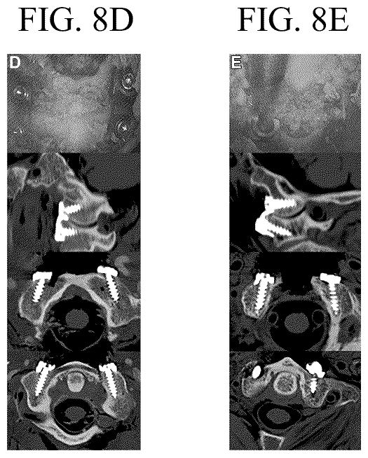

In one embodiment, the plates are customized for each cadaver. This particular approach was most conducive to the anatomical variability between specimens. Overall, relatively good screw accuracy was achieved with 85% optimally placed. It is likely that this number could be significantly higher for experienced operators with the requisite practice. To this end, a few considerations are worth discussing. First, we used neuronavigation-guidance for only 1 specimen, while the remaining 4 specimens were instrumented free-hand. In practice, the routine use of image-guidance, whether with neuronavigation or fluoroscopy, would be recommended in order to optimize safety and accuracy. This is particularly useful for certain challenging patient-specific anatomic features—for example, small condyles. In the single specimen in which our OC screws breeched, we noted the OCs to be unusually small. Regarding avoidance of injury to neurovascular structures, accurate screw placement is critical. With respect to the placing OC screws, a medial trajectory risks injury to the lower brainstem and/or vertebral arteries, while a superior trajectory risks injury to the hypoglossal nerve. For C1, a medial screw trajectory risks injury to the upper spinal cord or dural violation, while a lateral trajectory risks injury to the vertebral artery within the foramen trans-versarium (FIG. 7). In one embodiment, a preoperative CT angiogram is obtained to evaluate the positions of the pharyngeal carotid arteries, as approximately 4% of the population would be at risk of injury from elevation of the inverted U-shaped nasopharyngeal flap (IUNF). With regard to exposure, the IUNF provides superior visualization compared to that of the midline incision and can better support the intricacies of the procedure. Following IUNF elevation, the surgeon has an unencumbered view of the OCs and C1 lateral masses, affording sufficient exposure for instrumentation. Furthermore, the exposure provides a direct view of the O-C1 joints which can be decorticated to accommodate bone graft in order to promote bony fusion. While the IUNF provides an adequately wide exposure for the purpose of instrumentation, situations may arise that require additional lateral visualization. In these instances, partial resection of the torus tubarius would afford additional lateral visualization. However, it is not precisely known what effect this would have on the function of the Eustachian tube and middle ear.

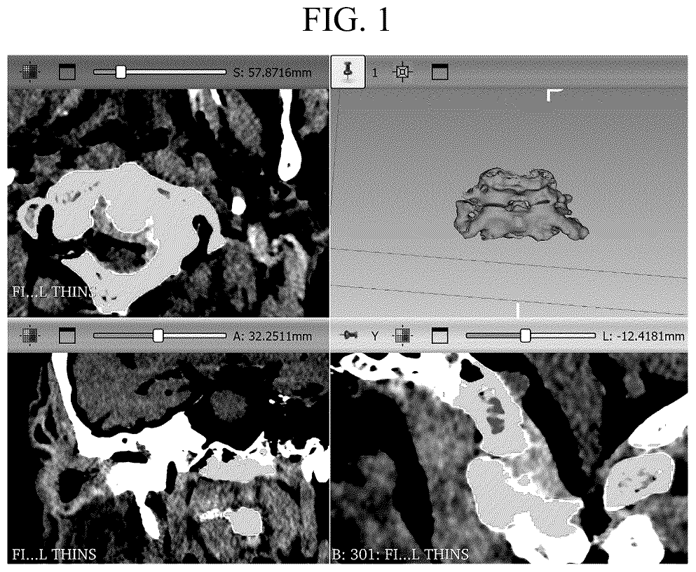

Referring to FIG. 1, thin-cut computed tomography scans were obtained for 5 cadaveric specimens. Image segmentation was used to reconstruct 3D models of each O-C1 joint complex. Using computer-aided design software, plates were custom-designed to span each O-C1 joint, sit flush onto the bony surface, and accommodate screws. The final models were 3D-printed in titanium. For implantation, specimens were held in pin-fixation and registered to neuronavigation. A rigid 0o endoscope was used for endonasal visualization. An inverted U-shaped nasopharyngeal flap was raised to expose the occipital condyles and C1. The plates were introduced and fixed with bone screws. Computed tomography scans were obtained to assess screw accuracy and proximity to critical neuro-vascular structures. Screw entry points and trajectories were recorded.

### Materials and Wound Healing

A few additional considerations are worth discussing. In one embodiment, the plate of the present invention was printed in a titanium alloy (Ti-6A1-4V), the most common alloy currently used in orthopedic surgery, which is known for its high yield strength, biocompatibility, corrosion-resistance, and rapid osseointegration. Other materials may be used based on their biocompatibility, osseointegration, and antimicrobial effect while limiting corrosion and failure. Useful materials include carbon fiber, polylactic acid, stainless steel alloys, commercially pure titanium, titanium alloys, ceramics, thermoplastics, polyethylene terephthalate (PET), fabric, silicone, polyurethane, silicone-polyurethane copolymers, polymeric rubbers, polyolefin rubbers, hydrogels, semi-rigid and rigid materials.

In various embodiments, the bone fixation fasteners of the present invention are bone screws, helical nails, distally expanding nails or distally expanding screws. In one embodiment, the bone fixation fasteners are bone screws. The present invention used a screw size of 4×17 mm in the examples. Selecting screw size involves a tradeoff between strength of the construct and risk of injury to nearby critical neurovascular structures (vertebral artery, spinal cord, cranial nerves, jugular vein, etc.). For example, while bicortical screw purchase would likely increase overall strength, the longer screws necessary would theoretically pose a higher risk of injury to those eloquent structures.

There are added considerations involving healing and the risks of wound-dehiscence or surgical site infection (SSI) following IUNF elevation and resuspension. While the hypothetical risk of postoperative SSI after endonasal instrumentation of the CVJ remains a concern, the data are limited on this topic as the totality of the literature consists of only 2 cases from a single case series. In that series, neither patient developed postoperative infectious complications during a mean follow-up time of 66 months. However, Woodhouse et al conducted a systematic analysis of SSI after transmucosal CVJ fixation, including 429 cases through the transoral approach and 2 cases through the endonasal approach—the reported rate of SSI was 1.8%. This did not statistically differ from the historically reported rates of SSI after posterior cervical fusion. Despite preliminary data indicating comparable rates of SSI between the transmucosal and posterior approaches, it is imperative to recognize the potentially devastating morbidity associated with such an infection. Rigorous investigation of methods and techniques aiming to minimize infectious complications will be necessary, including optimizing patterns of antibiotic prophylaxis. In addition, although not performed in the current study, resuspension of the IUNF can be accomplished using barbed, tabbed suture and a needle driver formatted for endonasal use.

### Variations and Indications

There are several potential indications for which this novel method of instrumentation could be applied. First, endonasal OCF after endonasal odontoidectomy would allow for a single-staged decompression and fixation. It should be noted, however, that fixation down to C2 may be necessary after odontoidectomy, as the majority of instability occurs at C1-2. Second, the application of endonasal OCF could be well-suited to address iatrogenic instability after endonasal tumor resection. As mentioned previously, the most common tumors of the lower clivus and anterior CVJ include chordomas, meningiomas, and chondrosarcomas. Select cases can be best approached through the endonasal corridor; however, those with extension into the OCs or atlanto-occipital joints often require their bony resection, and there is general consensus that OCF is required after resection of more than 75% of 1 condyle. In these cases, endonasal OCF would hold utility. Moreover, if the OC cannot accommodate a screw due to tumor invasion or bony resection, ventral fixation with a clival plate-screw construct has previously been described, and the EEA to the CVJ provides excellent visualization which could allow for this modification. Third, endonasal OCF could serve as a salvage option in degenerative pathology to limit further instability, such as with pseudoarthrosis of a failed posterior OCF or cranial settling after Chiari decompression. Lastly, endonasal access to the CVJ could be further modified and repurposed for fixation in trauma. For example, C1 osteosynthesis for Jefferson fractures has been described via the transoral approach, but not via the EEA. The method of the present invention could be accomplished equally as effectively via the EEA while avoiding the associated morbidity of the transoral approach. Additionally, an endonasal odontoid screw could theoretically be placed for stabilization of odontoid fractures in patients whose anatomy is not amenable to the traditional transcervical approach.

Endonasal instrumented fixation of the occipitocervical junction is both technically and anatomically feasible. The present method of endonasal OCF could potentially obviate the need for a staged posterior approach following endonasal procedures which destabilize the occipitocervical junction. The present invention may allow same-stage endonasal decompression/fixation. Additionally, it may offer minimally-invasive alternative to current methods of fixation, advancing our ability to treat pathology of the CVJ.

### EXAMPLES

### Example 1

Endonasal OCF was performed on 5 cadaveric specimens. The mean starting point for occipital condyle screws was 6.17 mm lateral and 5.38 mm rostral to the medial O-C1 joint. Mean axial and sagittal trajectories were 7.980 and 6.710, respectively. The mean starting point for C1 screws was 16.11 mm lateral to the C1 anterior tubercle and 6.39 mm caudal to the medial O-C1 joint. Mean axial and sagittal trajectories were 10.970 and −9.910, respectively.

Endonasal OCF is technically and anatomically feasible. The application of this technique may allow for same-stage endonasal decompression and fixation, offering a minimally invasive alternative to current methods of fixation and advancing surgeons' ability to treat pathology of the craniovertebral junction.

A recent cadaveric study described a method for endonasal atlanto-axial stabilization via transarticular screw fixation, and more recent cadaveric studies established the technical feasibility of endonasal screw placement into the occipital condyles (OCs) and C1 lateral masses. To date, however, no validated method exists for endonasal fixation across the CVJ, and the literature on investigational techniques is extremely limited. In this cadaveric feasibility study, we sought to build upon these concepts and design a plate-screw construct to be implanted via the endonasal corridor for OCF.

### Material and Methods

Five embalmed cadaveric specimens were utilized for anatomic dissection and implantation. The study was carried out in accordance with our institution's ethical standards and regulations. Institutional review board approval was neither required nor sought as the study did not constitute human subjects research. Appropriate consent was obtained for the publication of the cadaveric images.

### 3-Dimensional Segmentation of the O-C1 Joint

Thin-cut computed tomography (CT) scans (Revolution Apex CT System, GE HealthCare, Chicago, Illinois, USA) were obtained for each intact specimen. The associated Digital Imaging and Communications in Medicine files were imported into 3D Slicer v5.6.1 (Slicer), a free open-source software application used for medical image computing and visualization. The segment editor feature was used to construct a 3D model of the O-C1 joint by isolating the region of interest based on the expected density of bone (700e 2000 Hounsfield units) (FIG. 1). The resultant 3D model was then used to custom-design the plate.

### Design of a Customized 3-Dimensional O-C1 Plate-Screw Construct

A computer-aided design of the plate was generated using Blender (Blender Foundation, www.blender.org). First, the 3D model of the O-C1 joint was imported. Each plate was designed in accordance with the following specifications: to A) span the O-C1 joint and sit flush onto the bone surface, B) incorporate appropriately sized countersunk screw holes, and C) fit with ease through the endonasal corridor.

Size 4×17 mm self-drilling variable angle screws were used for fixation of the plate. The screw holes were sized such that the body of the screw could maintain sufficient degrees of freedom and designed such that the screw head would sit flush onto the counter-sink. The positions of the screw holes were designed to approximate prior descriptions of optimal starting points and trajectories for endonasal OC and C1 lateral mass screws, as reported by Forbes et al. The plates were designed with a thickness of 2.5 mm, consistent with several modem versions of anterior cervical discectomy and fusion plates.

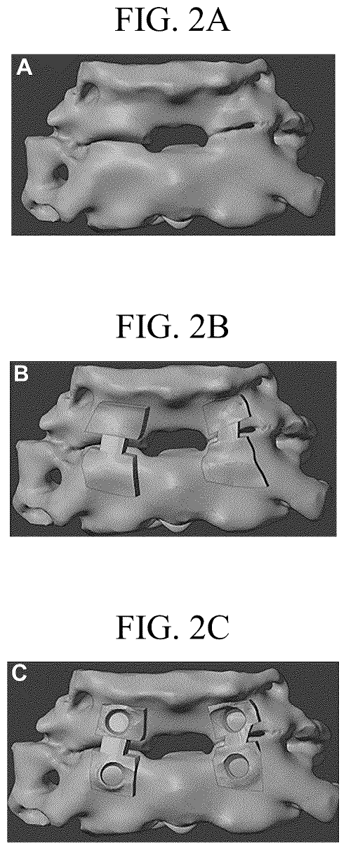

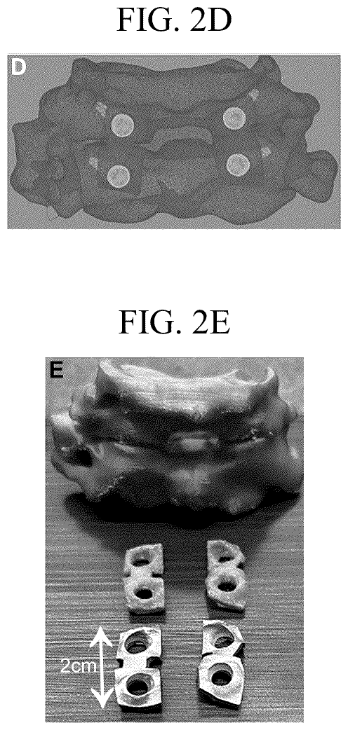

Once the above specifications were met, the plates (right and left) along with the corresponding O-C1 joint were 3D-printed in polylactic acid at our institution for prototyping. After final modifications were made, computer-aided design files of the plates were electronically sent to a third-party vendor (Proto Labs, Inc., Maple Plain, Minnesota, USA) to be 3D printed in a titanium alloy (Ti-6A1-4V) with direct metal laser sintering (FIG. 2).

### Setup, Exposure, and Implantation

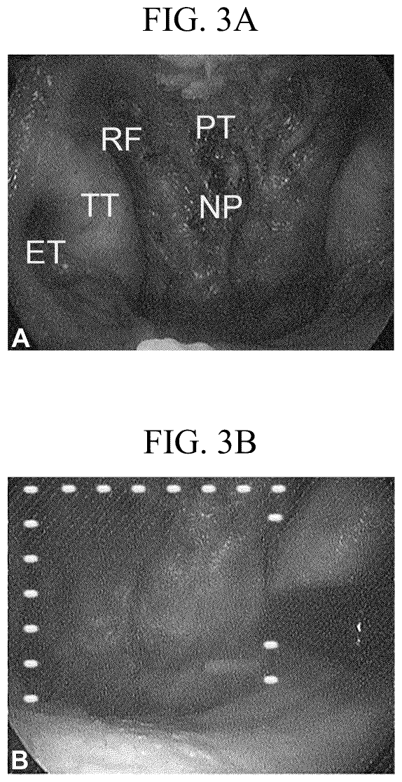

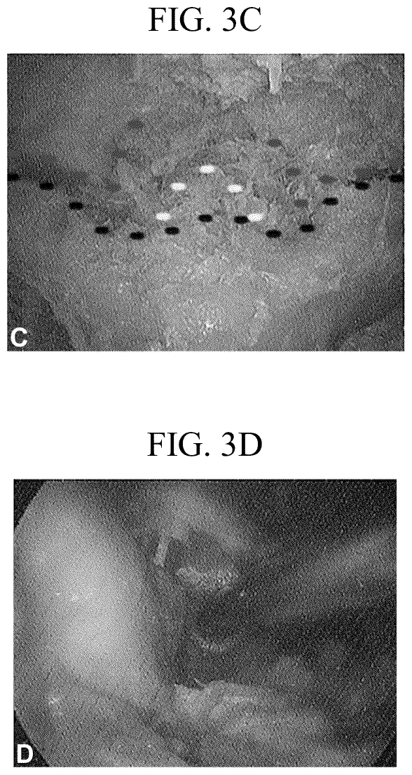

Implantation of the plates was performed in our microsurgical laboratory. Each specimen was immobilized in 3-point skeletal fixation with a Mayfield skull clamp (Integra LifeSciences, Princeton, New Jersey, USA). When neuronavigation was used, preoperative CT scans were uploaded to the Curve Navigation system (Brainlab, Munich, Germany) and the specimen was registered using surface-matching. A rigid 00 endoscope (Storz, El Segundo, California, USA) was used for endonasal visualization. As previously described in detail by Forbes et al, an inverted U-shaped nasopharyngeal flap (IUNF) was raised to expose the OCs, C1 arch, and O-C1 articulations. The O-C1 articulations may be decorticated at this point and later packed with autograft/allograft to facilitate arthrodesis. Each plate is then introduced and properly positioned onto the bony surface. Pilot holes are drilled and screws are then introduced endonasally with a driver (Medtronic, Minneapolis, Minnesota, USA). Each screw is driven into place, with or without neuronavigation-guidance, until tight and flush on the plate, (FIG. 3). Thin-cut CT scans are obtained postoperatively to evaluate screw accuracy and proximity to critical neurovascular structures (vertebral artery, hypoglossal nerve, C1 nerve root, spinal cord, etc.). We defined screw placement as “optimal” if all parts of the screw were within the OC or the C1 lateral mass without a breach. If any part of the screw breeched the

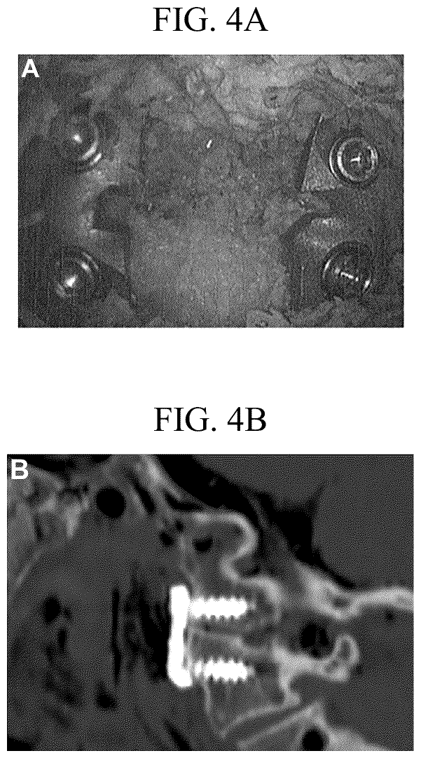

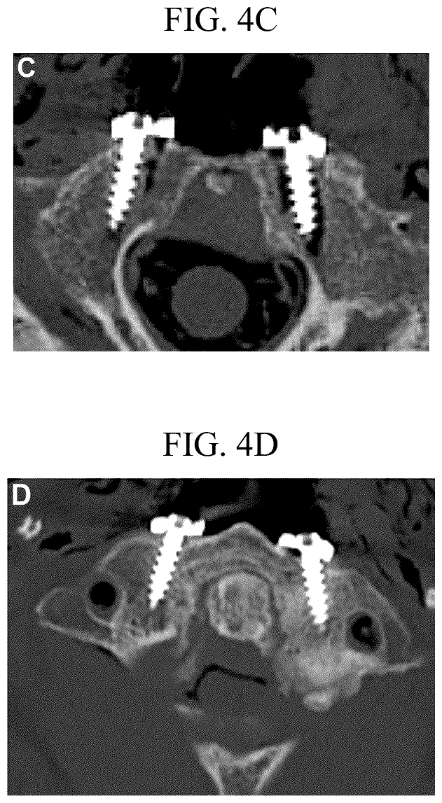

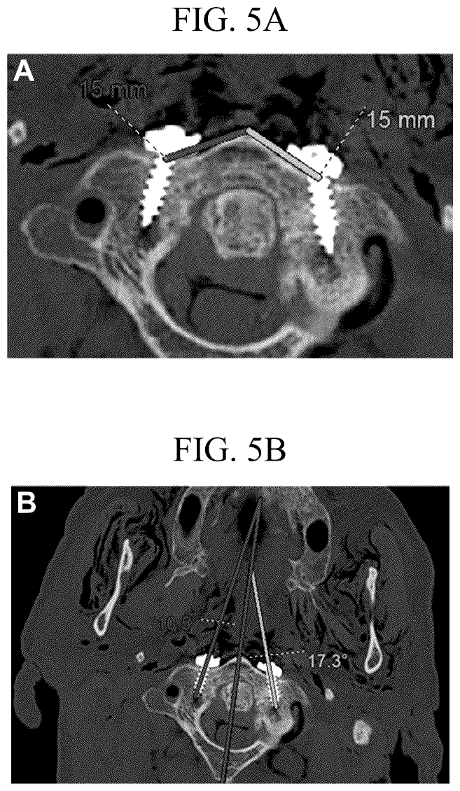

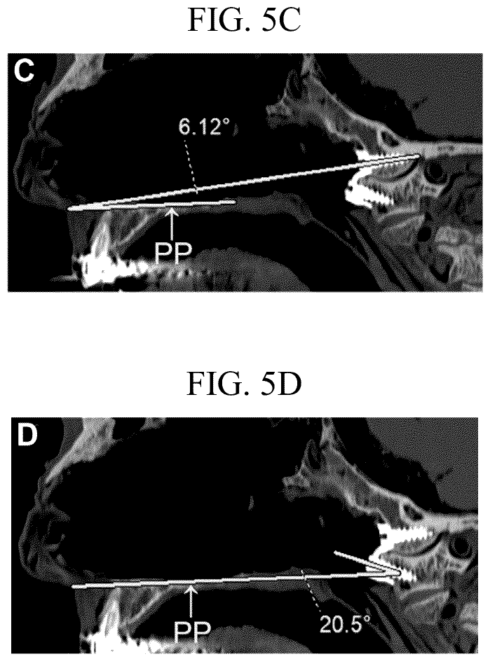

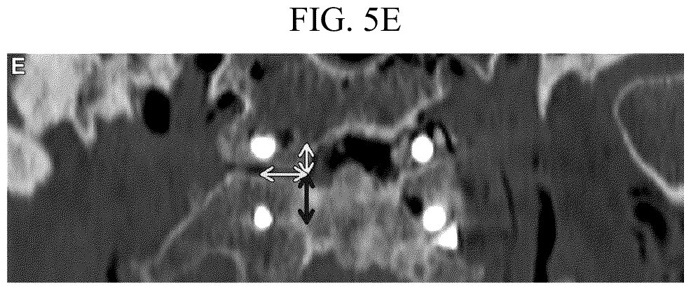

outer cortex of bone medially/laterally, violated the joint space, or came into contact with any neurovascular structure, we deemed this “suboptimal” (FIG. 4). In addition, the following parameters were measured and recorded using our institution's picture archiving and communication system platform (McKesson Technology Solutions, Irving, Texas, USA): 1) lateral site-of-entry, as measured from the anterior tubercle of C1 (for C1) and the medial O-C1 joint-line (for OC), 2) rostral/caudal site-of-entry, as measured from the medial O-C1 joint, and 3) screw angulation in the axial plane with respect to the midline and in the sagittal plane with respect to the palatal plane (FIG. 5).

O-C2 Fixation with C1-2 Transarticular Screw

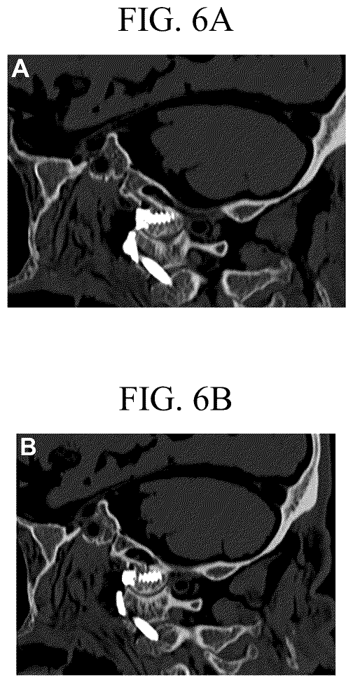

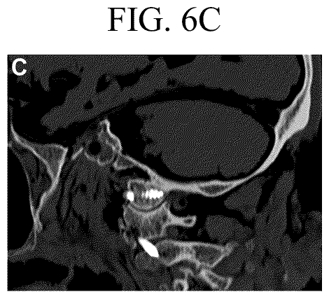

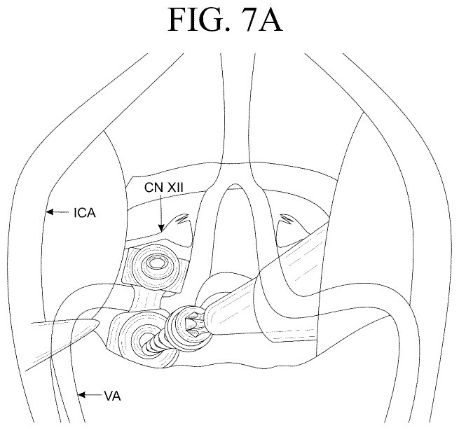

On 1 side of a single cadaver, we sought to demonstrate the feasibility of extending fixation to C2 by placing a C1-2 trans-articular screw instead of a C1 lateral mass screw. We chose to use a longer (4×30 mm) self-tapping double lead screw in order to ensure adequate purchase into the C2 pars interarticularis (FIG. 6). Because of the technically challenging inferior trajectory required for screw placement, we used an awl and a screw-driver with a U-joint which permitted sufficient angulation (Depuy Synthes, Raynham, Massachusetts, USA).

### Statistical Analysis

Statistical analysis was performed using Microsoft Excel and GraphPad Prism (Prism v10.1.1, GraphPad Software, Boston, Massachusetts, USA). Means and standard deviations were calculated. We did not statistically compare means between groups due to small sample size and lack of useful information this would have given us in the context of our study design.

Endoscopic endonasal OCF was successfully performed on 5 cadaveric specimens. Of the 10 OC screws placed, 8 were optimal and 2 were suboptimal. The 2 suboptimal screws were placed on the left and right of the same specimen and both breached superomedially, coming into near contact with the hypoglossal canals. The mean starting point for all OC screws placed was 6.17 mm lateral and 5.38 mm rostral to the medial O-C1 joint. Mean axial and sagittal trajectories were 7.980 and 6.710, respectively. Of the 9 C1 lateral mass screws placed, 8 were optimal and 1 was suboptimal. The suboptimal screw breached the O-C1 joint space superomedially. The mean starting point for all C1 screws placed was 16.11 mm lateral to the C1 anterior tubercle and 6.39 mm caudal to the medial O-C1 joint. Mean axial and sagittal trajectories were 10.970 and −9.910, respectively. One screw was placed from the anterior arch of C1 to the pars interarticularis of C2 (anterior C1-2 transarticular screw). Starting point and trajectory data are reported separately for optimal and suboptimal screws in Table 1.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

TABLE 1
Trajectories and Starting Points for Screws Placed
into the Occipital Condyles and Cl Lateral Masses

| | Sagittal | Axial | Lateral Site-of- | Rostral Site-of- | Caudal Site-of- |
|---|---|---|---|---|---|
| Level | Trajectory (°) | Trajectory (°) | Entry (mm)* | Entry (mm)† | Entry (mm)‡ |
| OC (all) | 6.71 ± 4.8 | 7.98 ± 6.7 | 6.17 ± 1.7 | 5.38 ± 2.0 | |
| | (−5.7-12.1) | (−6.8-19.2) | (2.9-8.4) | (1.2-7.3) | |
| Optimal (n = 8) | 6.02 ± 5.2 | 9.91 ± 4.7 | 6.2 ± 1.9 | 5.08 ± 2.1 | |
| Suboptimal (n = 2) | 9.48 ± 1.0 | 0.26 ± 10.0 | 6.05 ± 1.3 | 6.6 ± 0.8 | |
| C1(all) | −9.91 ± 6.0 | 10.97 ± 7.9 | 16.11 ± 1.8 | | 6.39 ± 2.2 |
| | (−18.4-2.5) | (−6.9-18.5) | (14-19) | | (2.8-8.7) |
| Optimal (n = 8) | −11.46 ± 4.1 | 13.21 ± 4.4 | 16.13 ± 2.0 | | 6.84 ± 1.8 |
| Suboptimal (n = 1) | 2.45 | −6.89 | 16 | | 2.8 |

OC, occipital condyle.
*Measured from the anterior tubercle of Cl (for Cl) and the medial 0-Cl joint-line (for OC).
†Measured as the rostral distance from the medial 0-Cl joint.
‡Measured as the caudal distance from the medial 0-Cl joint.

What is claimed is:

1. An implant system for the fusion of the occipitocervical (O-C1) joint comprising:
a. a pair of plates comprising a right plate and a left plate;
i. the right plate defining at least a first right plate opening and a second right plate opening;
ii. the left plate defining at least a first left plate opening and a second left plate opening; and
b. at least four bone fixation fasteners;
i. a first fastener disposed with the first right plate opening;
ii. a second fastener disposed with the first left plate opening;
iii. a third fastener disposed with the second right plate opening;
iv. a fourth fastener disposed with the second left plate opening; wherein the first fastener is engageable with the right plate and capable of fixing with the occipital condyle, the second fastener is engageable with the left plate and capable of fixing with the occipital condyle, the third fastener is engageable with the right plate and capable of fixing with the C1 lateral mass, and the fourth fastener is engageable with the left plate and capable of fixing with the C1 lateral mass.

2. The implant system of claim 1 wherein the pair of plates comprise a material selected from the group consisting of carbon fiber, polylactic acid, stainless steel alloys, commercially pure titanium, titanium alloys, ceramics, thermoplastics, polyethylene terephthalate (PET), fabric, silicone, polyurethane, silicone-polyurethane copolymers, polymeric rubbers, polyolefin rubbers, hydrogels, semi-rigid and rigid materials.

3. The implant system of claim 1 wherein the pair of plates comprise titanium alloy.

4. The implant system of claim 1 wherein the bone fixation fasteners are selected from the group consisting of bone screws, helical nails, distally expanding nails, and distally expanding screws.

5. The implant system of claim 1 wherein the bone fixation fasteners are bone screws.

6. The implant system of claim 1 wherein the right plate and left plate each have an average thickness of 2.5 mm.

7. An atlanto-occipital fusion method for fixating a 0-C1 joint, comprising:
a. preparing an endonasal surgical path using a binostril approach comprising:
i. removing the inferior portion of the posterior septum,
ii. performing a myomucosal flap incision to create a flap,
iii. retracting the flap to expose the O-C1 joint,
b. bilaterally decorticating the O-C1 joint, and
c. inserting a plate into each side of the decorticated 0-C1 joint;
wherein the plate comprises a plate from the implant system of claim 1.

8. The atlanto-occipital fusion method of claim 7 wherein the pair of plates of the implant system comprise a material selected from the group consisting of carbon fiber, polylactic acid, stainless steel alloys, commercially pure titanium, titanium alloys, ceramics, thermoplastics, polyethylene terephthalate (PET), fabric, silicone, polyurethane, silicone-polyurethane copolymers, polymeric rubbers, polyolefin rubbers, hydrogels, semi-rigid and rigid materials.

9. The atlanto-occipital fusion method of claim 7 wherein the pair of plates of the implant system comprise titanium alloy.

10. The atlanto-occipital fusion method of claim 7 wherein the bone fixation fasteners of the implant system are selected from the group consisting of bone screws, helical nails, distally expanding nails, and distally expanding screws.

11. The atlanto-occipital fusion method of claim 7 wherein the bone fixation fasteners of the implant system are bone screws.

12. The atlanto-occipital fusion method of claim 7 wherein the right plate and left plate of the implant system each have an average thickness of 2.5 mm.
